(19) United States
(12) Patent Application Publication
(10) Pub. No.: US 2018/0003663 A1
(43) Pub. Date: Jan. 4, 2018

(54) CHEMICAL SENSOR, AND CHEMICAL SUBSTANCE DETECTION METHOD AND DEVICE

(71) Applicant: Hitachi, Ltd., Tokyo (JP)

(72) Inventors: Norifumi KAMESHIRO, Tokyo (JP); Hiromasa TAKAHASHI, Tokyo (JP); Sanato NAGATA, Tokyo (JP); Shirun HO, Tokyo (JP)

(21) Appl. No.: 15/593,421

(22) Filed: May 12, 2017

(30) Foreign Application Priority Data
Jun. 29, 2016 (JP) 2016-128640

(51) Int. Cl. G01N 27/22; H01L 29/16; G01N 33/543; H01L 29/20; H01L 29/161

(52) U.S. Cl. CPC G01N 27/226; H01L 29/2003; H01L 29/161; G01N 27/227; G01N 33/54393; G01N 33/5438; H01L 29/1608

(57) ABSTRACT

There is a provided a chemical sensor that includes a semiconductor substrate of a first conductivity type, a first electrode that is formed on a front surface of the semiconductor substrate, a second electrode that is disposed to face the first electrode in a vertical direction, a flow path in which a liquid or a gas can flow between the first electrode and the second electrode, and a chemical substance capturing portion that is disposed in at least a partial region between the first electrode and the second electrode in the flow path, and bonded with a predetermined chemical substance, and in which a distance between the first electrode and the second electrode is set to be 2 nm or more and 200 nm or less, and a change in dielectric constant between the first electrode and the second electrode is detected.

### CLAIM OF PRIORITY

The present application claims priority from Japanese application serial no. JP 2016-128640, filed on Jun. 29, 2016, the content of which is hereby incorporated by reference into this application.

### BACKGROUND OF THE INVENTION

### Field of the Invention

The present invention relates to a chemical sensor using a semiconductor substrate, a manufacturing method thereof, and chemical substance detection device and method.

### Background Art

There are JP-A-2010-160151 and JP-A-2008-111854 as background art of the field of the invention. In JP-A-2010-160151, a chemical sensor that uses an antibody probe on an electrode formed on a nonconductive substrate to detect a presence or absence of an antigen by an electric sensing method is described. A conductivity promoting molecule that promotes the conductivity of the electric sensing method is disposed on the electrode and an antibody layer is disposed therethrough. Therefore, it is possible to amplify the signals of the electric sensing type sensor.

In addition, in JP-A-2008-111854, as a molecular recognition sensor formed on a semiconductor substrate, a sensor that detects a change in electrostatic capacitance of a substrate when a recognition target molecule is captured by a recognition material portion by using a change in photocurrent is described.

### SUMMARY OF THE INVENTION

A chemical sensor that detects a specific chemical substance is configured to include a detection unit that detects a chemical substance and an output unit that outputs a result thereof. A signal outputted when a trace amount of chemical substance is detected by the detection unit is small and buried in a noise signal, and is erroneously detected in some cases.

For example, as a method for improving erroneous detection due to the above-described minute signal, there are techniques described in JP-A-2010-160151 and JP-A-2008-111854. In the sensor disclosed in JP-A-2010-160151, conductivity between electrodes can be improved by using the conductivity promoting molecule, and a signal can be amplified to a level measurable by a measuring instrument. In addition, in the sensor disclosed in JP-A-2008-111854, a minute detection signal is amplified by detecting a photocurrent generated by light irradiation.

However, in order to amplify the signal, it is necessary to add a configuration and complicate the structure, which leads to an increase in a size and a cost of the device. Therefore, it is necessary to study a chemical sensor which does not require signal amplification and does not cause erroneous detection.

According to an aspect of the invention for solving the above-described problems, there is provided a chemical sensor that includes a semiconductor substrate of a first conductivity type, a first electrode that is formed on a front surface of the semiconductor substrate, a second electrode that is disposed to face the first electrode in a vertical direction, a flow path in which a liquid or a gas can flow between the first electrode and the second electrode, and a chemical substance capturing portion that is disposed in at least a partial region between the first electrode and the second electrode in the flow path, and bonded with a predetermined chemical substance, and in which a distance between the first electrode and the second electrode is set to be 2 nm or more and 200 nm or less, and a change in dielectric constant between the first electrode and the second electrode is detected.

According to another aspect of the invention, there is provided a chemical substance detection method that includes using a semiconductor substrate, a first electrode that is formed on a front surface of the semiconductor substrate, a second electrode that is disposed to face the first electrode in a vertical direction, and a chemical substance capturing portion that is disposed in at least a partial region between the first electrode and the second electrode and bonded with a predetermined chemical substance, setting a distance between the first electrode and the second electrode to be 100 times or less the size of the chemical substance, supplying a gas or a liquid containing the chemical substance between the first electrode and the second electrode, detecting a change in dielectric constant between the first electrode and the second electrode by capturing the chemical substance in the chemical substance capturing portion, and detecting the chemical substance based on the change in the dielectric constant.

According to another aspect of the invention, there is provided a chemical substance detection device that includes a plurality of chemical sensors, each of which includes a semiconductor substrate, a first electrode that is formed on a front surface of the semiconductor substrate, a second electrode that is disposed to face the first electrode in a vertical direction, a flow path in which a liquid or a gas can flow between the first electrode and the second electrode, a chemical substance capturing portion that is disposed in at least a partial region between the first electrode and the second electrode in the flow path, and bonded with a predetermined chemical substance, and in which a distance between the first electrode and the second electrode is set to be 2 nm or more and 200 nm or less, and in which one electrode of the first or second electrode of the chemical sensor is connected to ground, the other electrode of the chemical sensor is connected to a detection system, and the detection system includes a power supply for applying a voltage and a current meter, and detects an electrostatic capacitance between the first electrode and the second electrode.

The chemical sensor according to the invention can detect the change in the dielectric constant between facing electrodes without requiring signal amplification. The problems, configurations, and effects other than those described above will be clarified by the description of the embodiments below.

### BRIEF DESCRIPTION OF THE DRAWINGS

FIGS. 1A and 1B are conceptual diagrams illustrating a main part of a chemical sensor according to Example 1.

FIG. 2A is a cross-sectional view of the main part illustrating a manufacturing process of the chemical sensor according to Example 1.

FIG. 2B is a cross-sectional view of the main part illustrating the manufacturing process of the chemical sensor according to Example 1 (continued).

FIG. 2C is a cross-sectional view of the main part illustrating the manufacturing process of the chemical sensor according to Example 1 (continued).

FIG. 2D is a cross-sectional view of the main part illustrating the manufacturing process of the chemical sensor according to Example 1 (continued).

FIG. 2E is a cross-sectional view of the main part illustrating the manufacturing process of the chemical sensor according to Example 1 (continued).

FIG. 3A is a cross-sectional view of a main part illustrating a structure of the chemical sensor according to Example 1.

FIG. 3B is a plan view of the main part illustrating the structure of the chemical sensor according to Example 1.

FIG. 3C is a cross-sectional view of the main part illustrating the structure of the chemical sensor according to Example 1.

FIG. 4 is a cross-sectional view of a main part illustrating another modification example of a structure of the chemical sensor according to Example 1.

FIG. 5 is a cross-sectional view of a main part illustrating another modification example of the structure of the chemical sensor according to Example 1.

FIGS. 6A and 6B are cross-sectional views of main parts illustrating still another modification example of the structure of the chemical sensor according to Example 1.

FIG. 7 is a cross-sectional view of a main part illustrating still another modification example of the structure of the chemical sensor according to Example 1.

FIG. 8 is a cross-sectional view of a main part illustrating still another modification example of the structure of the chemical sensor according to Example 1.

FIG. 9 is a cross-sectional view of a main part illustrating still another modification example of the structure of the chemical sensor according to Example 1.

FIG. 10 is a cross-sectional view of a main part illustrating still another modification example of the structure of the chemical sensor according to Example 1.

FIG. 11A is a cross-sectional view of a main part illustrating a manufacturing process of a chemical sensor according to Example 3.

FIG. 11B is a cross-sectional view of the main part illustrating the manufacturing process of the chemical sensor according to Example 3 (continued).

FIG. 12 is a schematic diagram illustrating a configuration of a chemical substance detection device according to Example 4.

FIG. 13 is a schematic diagram illustrating another modification example of the configuration of the chemical substance detection device according to Example 4.

FIG. 14 is a schematic diagram illustrating another modification example of the configuration of the chemical substance detection device according to Example 4.

### DETAILED DESCRIPTION OF THE INVENTION

In the following embodiments, when necessary for convenience, the description will be divided into a plurality of sections or embodiments, but these are not unrelated to each other, and one is related to a modification example, detail, and a supplementary explanation of a portion or all of the other, except for a case of being expressly stated in particular.

In addition, in the following embodiments, when the number of elements (including number, numerical value, amount, range, and the like) is referred to, the number of elements is not limited to a specific number thereof, and may be the specific number or more or less, except for a case of being expressly stated in particular and a case of being obviously limited to the specific number in principle.

In addition, in the following embodiments, it is needless to say that the configuration elements thereof (including element step and the like) are not necessarily indispensable, except for a case of being expressly stated in particular and a case where it is obviously considered indispensable in principle.

In addition, when “comprising A”, “consisting of A”, “having A”, and “including A” are referred to, it is needless to say that these do not exclude other elements, except for a case where only the element thereof is expressly stated in particular. Similarly, in the following embodiments, when a shape and a positional relation of configuration elements are referred to, these include substantially similar or similar to the shape thereof, except for a case of being expressly stated in particular and a case where it is obviously considered not to be so in principle. This fact is similar to the above numerical values and ranges.

In addition, in all the drawings for explaining the following embodiments, those having the same functions are denoted by the same reference numerals in principle, and the repeated descriptions thereof will be omitted. Hereinafter, the embodiment will be described in detail with reference to the drawings.

A chemical sensor for detecting a specific chemical substance is configured to include a detection unit for detecting the chemical substance and an output unit for outputting the result thereof. In order to output a signal change when a trace amount of chemical substance is detected in the detection unit, signal amplification is required in the output unit. However, in order to amplify the signal, there is a problem that it is necessary to add a configuration and complicate a structure, which leads to an increase in a size and a cost of the device. Therefore, consideration of the chemical sensor that does not require the signal amplification and that does not cause erroneous detection is required. In the chemical sensor of an example described below, when detecting a trace amount of the chemical substance in the detection unit, a change rate of the signal is increased, and the structure is such that signal amplification is not required.

An example of a chemical sensor includes a first electrode that is formed on a front surface of the semiconductor substrate, a second electrode that is disposed to face the first electrode in a vertical direction, a flow path in which a liquid or a gas can flow between the first electrode and the second electrode, and a chemical substance capturing portion that is disposed in at least a partial region between the first electrode and the second electrode in the flow path, and has high bonding property with a predetermined chemical substance, and in which a distance between the first electrode and the second electrode is smaller than a predetermined ratio with respect to a size of a molecule of the predetermined chemical substance, and a change in dielectric constant between the first electrode and the second electrode is detected when the predetermined chemical substance is bonded to the chemical substance capturing portion.

### Example 1

Structure of Chemical Sensor

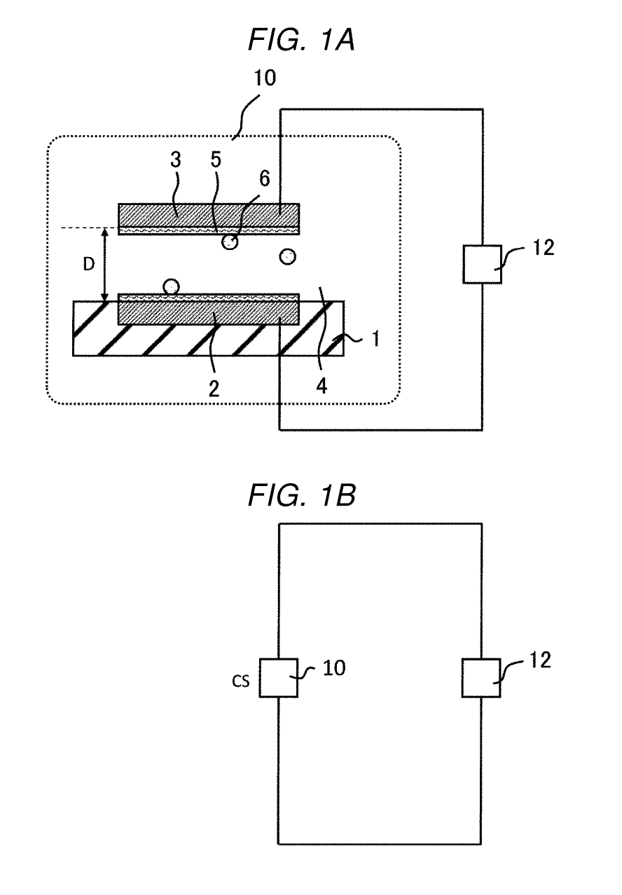

The structure of the chemical sensor according to Example 1 will be described with reference to FIGS. 1A and 1B. FIGS. 1A and 1B are conceptual diagrams illustrating the structure of the chemical sensor according to Example 1. As illustrated in the sectional shape in FIG. 1A, in a chemical sensor 10 according to Example 1, a first electrode 2 which is an n⁺-type semiconductor region formed by an impurity implantation of high concentration on the front surface of a semiconductor substrate 1 of p-type silicon (Si), a second electrode 3 formed of n⁺ polycrystalline silicon disposed to face the first electrode 2 in a vertical direction, and a flow path 4 for flowing a liquid or a gas between the first electrode 2 and the second electrode 3 is formed. A chemical substance capturing portion 5 having a high bonding property with a predetermined chemical substance 6 is formed in at least a portion of the first electrode 2 and the second electrode 3 in the flow path.

The chemical substance capturing portion 5 is a silane coupling agent having an organic functional group on the front surface, or a synthetic molecule prepared by further molecular imprinting on the front surface of the silane coupling agent, or a modified antibody. A distance D between the first electrode 2 and the second electrode 3 is set to be a value smaller than a predetermined ratio (approximately 100 times) with respect to the size of the molecule of the predetermined chemical substance 6 to be detected. Furthermore, the first electrode 2 and the second electrode 3 are connected to a detection system 12 including a power supply.

A connection relationship between a main part of the chemical sensor 10 and a detection system including the power supply will be described with reference to FIG. 1A. As illustrated in FIG. 1A, the first electrode 2 and the second electrode 3 are connected to the detection system 12 including the power supply via a metal wiring layer (not illustrated) or the like. The detection system 12 detects a change in capacitance between the first electrode 2 and the second electrode 3. As a specific configuration, a measurement method used for capacitance measurement of an ordinary capacitor may be used, and may be selected according to characteristics of the object to be evaluated, such as (1) measuring the current at the time using a low frequency voltage that can pass through the capacitance, (2) measuring a current change at the time by applying a small potential difference, (3) measuring the change in the voltage at the time by applying a current pulse, and (4) measuring the current change by superimposing the DC voltage on the AC voltage. In addition, dependency on the frequency of the AC voltage is measured as required in some cases.

The capacitance is derived from an equation of impedance Z

V=Z·I

Z=R+jwL+1/(jwC)

(R is a resistance, j is an imaginary unit, L is an inductance, C is a capacitance, w=2πf is an AC angular frequency of a frequency f, V is a voltage measured by a voltage meter, and I is a current measured by a current meter). A detection principle of the example is basically to detect the change of C in the equation of impedance Z with R and L fixed.

The capacitance C between plate electrodes is applied by C=∈S/D (∈ is a dielectric constant of the substances between the electrodes and S is an area of the plate electrode).

In the example, the distance D is set to be a value smaller than approximately 100 times the size of the molecule of the predetermined chemical substance 6 to be detected. In this manner, the proportion of the chemical substance occupying the space between the electrodes increases, so that the dielectric constant between the electrodes greatly changes by the chemical substance 6 captured in the chemical substance capturing portion 5. As a result, the rate of change of ∈ increases and a large change rate of C is obtained.

FIG. 1B is an equivalent circuit diagram in which the chemical sensor 10 of FIG. 1A is rewritten with a simplified symbol. CS in the figure is an abbreviation for the chemical sensor.

“−” and “+” are symbols indicating relative impurity concentrations of n-type or p-type conductivity, for example, the impurity concentration of the n-type impurity increases in the order of “n⁻”, and “n⁺”, and the impurity concentration of the p-type impurity increases in the order of “p⁻”, “p”, and “p⁺”.

Manufacturing Process of Chemical Sensor

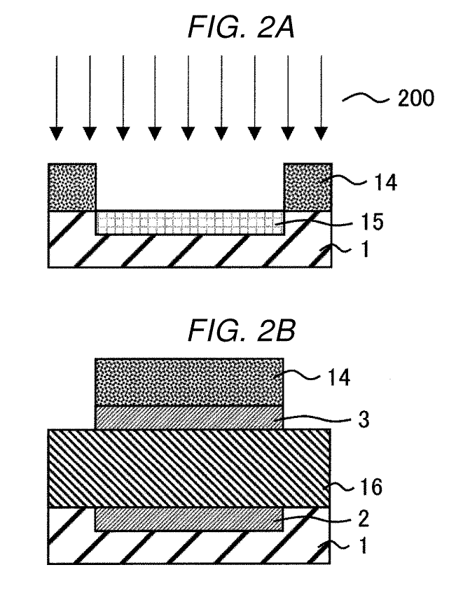

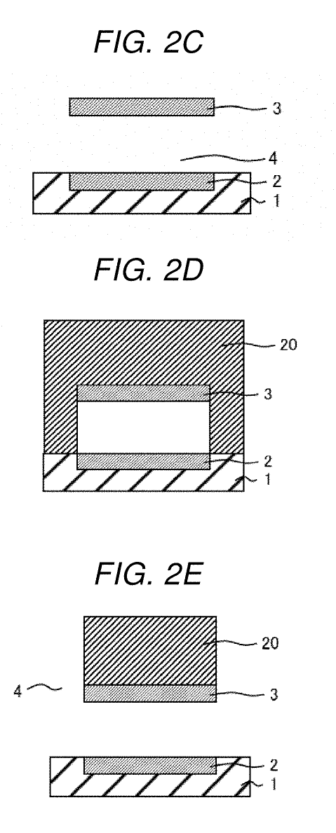

In the chemical sensor 10 having the configuration illustrated in FIGS. 1A and 1B, since the rate of change in the capacitance between the first electrode 2 and the second electrode 3 caused by the chemical substance 6 is large, detection with high sensitivity becomes possible. Such a configuration can be manufactured by a semiconductor process to which a semiconductor technology is applied. A manufacturing method of the chemical sensor according to Example 1 will be described in order of processes with reference to FIGS. 2A to 2E. Each figure is a cross-sectional view of the main part illustrating a manufacturing process of the chemical sensor according to Example 1.

In a process of FIG. 2A, the semiconductor substrate 1 of p-type Si is first prepared. The Si semiconductor substrate 1 has different specifications of various surface orientations and resistivity, but any specifications do not matter as long as the specifications can be used for ordinary semiconductor process applications.

Next, a mask material 14 is formed on the upper surface of the p-type Si semiconductor substrate 1, and the mask material 14 is patterned by a photolithography technique. Any other materials can be used as the mask material as long as the material is to be the mask at the time of ion implantation, for example, such as silicon oxide (SiO₂), silicon nitride (Si₃N₄), resist material or the like formed by chemical vapor deposition (CVD) method.

Subsequently, an n-type impurity 200 is ion-implanted into the upper surface of the p-type Si semiconductor substrate 1 exposed from the patterned mask material 14, so that an n⁺-type semiconductor region 15 is formed on the upper surface of the p-type Si semiconductor substrate 1. For an ion implantation condition, for example, phosphorus (P) is set in the range of 3 to 50 keV and 1 to 5×10¹⁵ cm⁻².

After removal of the mask material 14, an activation process (annealing) of implanted impurity is performed, so that the n⁺-type semiconductor region 15 becomes the first electrode 2. The ion implantation condition is adjusted so that the impurity concentration at this time is approximately 1 to 5×10²⁰ cm⁻³.

FIG. 2B illustrates a process after forming the first electrode 2. A spacer 16 is formed on the upper surface of the p-type Si semiconductor substrate 1. The material of the spacer may be any material which is selectively etched with hydrofluoric acid, for example, SiO₂ formed by a CVD method.

After forming the spacer 16, a film serving as a material of the second electrode, for example, n⁺ polycrystalline silicon by the CVD method is formed on the entire upper surface of the spacer 16, the mask material 14 is formed on the upper surface thereof, and the mask material 14 is patterned by a photolithography technique. Thereafter, a layer serving as the material of the second electrode is processed to form the second electrode 3.

FIG. 2C illustrates a process after forming the second electrode 3 by removing the mask material 14. The exposed spacer 16 is selectively etched with hydrofluoric acid to form the flow path 4 between the electrodes.

FIG. 2D illustrates a process after forming the flow path 4 between the electrodes. A protective film 20 is formed on the second electrode 3 and the semiconductor substrate 1. As the protective film 20, in order to prevent the film from flowing and accumulating in the flow path 4 between the formed electrodes, for example, a physical vapor deposition (PVD), or SiO₂ or Si₃N₄ formed by the CVD method using a condition with poor step coverage is used.

FIG. 2E illustrates a process after forming the protective film 20. The protective film 20 is etched so as to leave the upper portions of the second electrode 3 and the second electrode 3. The space on the side of the second electrode 3 and the protective film 20 removed by etching functions as the flow path 4 of a medium for transporting the chemical substance. Although not illustrated in the drawing, a partition wall for restricting the flow path by leaving a portion of the protective film 20 can be provided.

Thereafter, after the chemical substance capturing portion 5 is formed on a side wall of the flow path including the front surfaces of the first electrode 2 and the second electrode 3, the chemical substance capturing portion 5 at an unnecessary place is removed as required.

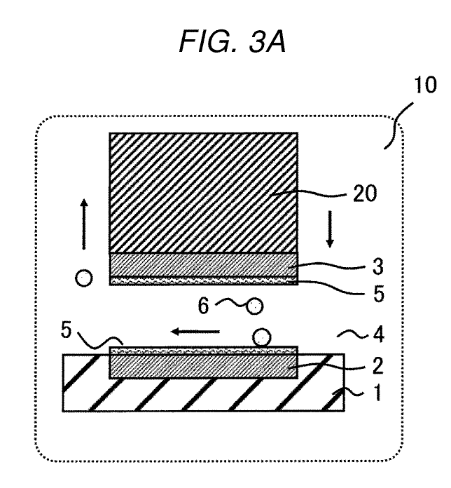

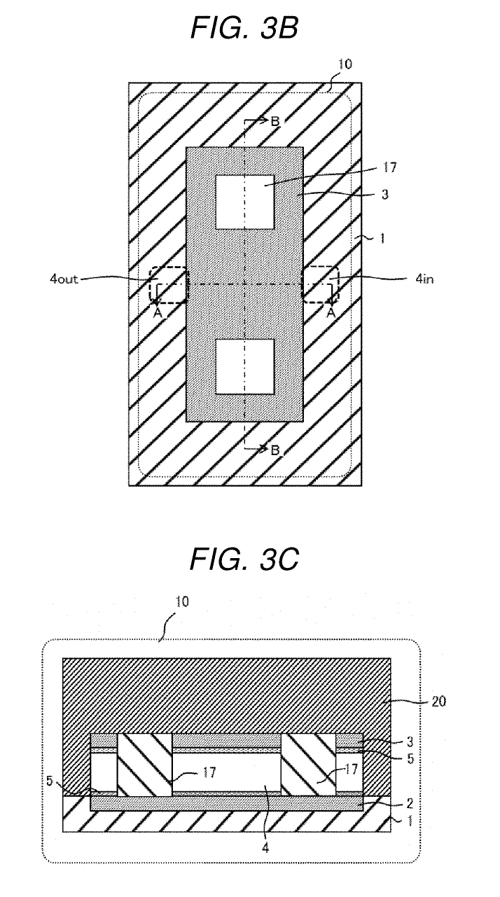

FIGS. 3A to 3C are schematic views of the chemical sensor 10 completed by the process of FIGS. 2A to 2E. FIG. 3A is a cross-sectional view after completing the process of FIG. 2E. The gas or liquid carrying the chemical substance 6 is transported, for example, as illustrated by an arrow in FIGS. 3A to 3C, and the chemical substance 6 is captured by the chemical substance capturing portion 5 between the electrodes.

FIG. 3B is a top view of the configuration of FIG. 3A. The cross-sectional view of FIG. 3A illustrates a cross section A-A of FIG. 3B. In order to form the flow path 4, a lower part of the second electrode 3 of the chemical sensor 10 illustrated in FIG. 3B is a gap obtained by removing the spacer 16. For the description, the protective film 20 is omitted from illustration.

In the example, in order to hold the second electrode 3, a columnar structure 17 is disposed at a predetermined position. In the example of FIG. 3B, the second electrode 3 is formed around the columnar structure 17, and the hidden invisible chemical substance capturing portion 5 and the first electrode 2 have the same planar shape as the second electrode 3.

In addition, FIG. 3B schematically illustrates an inlet 4in and an outlet 4out of the flow path 4. The flow path 4 in the direction perpendicular to the semiconductor substrate 1 is formed by removing the protective film 20 up to the semiconductor substrate 1 by the process of FIG. 2E. The shape and size of the flow path 4 in the vertical direction can be arbitrarily formed by etching of the protective film 20 in FIG. 2D. In the drawing, the inlet 4in and the outlet 4out are illustrated relatively small, but it is generally preferable to form as large as possible and leave necessary portion such as partition wall of the flow path.

In the configuration of FIGS. 3A to 3C, the number of the columnar structures 17 is two, but three or more columnar structures 17 may be provided by extending the sensor portion such as the second electrode 3 further in a B-B direction.

FIG. 3C illustrates a cross section B-B of FIG. 3B. As a material of the columnar structure 17, it is necessary to be a material resistant to etching when the spacer 16 is removed. For example, the material is an insulator which is hardly etched with hydrofluoric acid, and uses, for example, silicon nitride (Si₃N₄) or tantalum pentoxide (Ta₂O₅). Although the process is not limited, it may be formed after forming the film to be the material of the second electrode, and before removing the spacer 16. For example, after removing the mask material 14 in the state of FIG. 2B, a hole penetrating the second electrode 3 and the spacer 16 to reach the first electrode 2 or the semiconductor substrate 1 is formed, and an insulator is accumulated in the hole. The columnar structure 17 is formed, so that a mechanical strength of the second electrode 3 can be improved.

Modification Example of Chemical Sensor and Manufacturing Method of Chemical Sensor

Next, a modification example of the chemical sensor according to Example 1 and a manufacturing method of the chemical sensor will be described.

(1) In the example of FIGS. 3A to 3C, the chemical sensor configured to include a pair of first electrode and second electrode disposed to face each other is used, but the chemical sensor configured to include a plurality of pairs of first electrode and second electrode may be used.

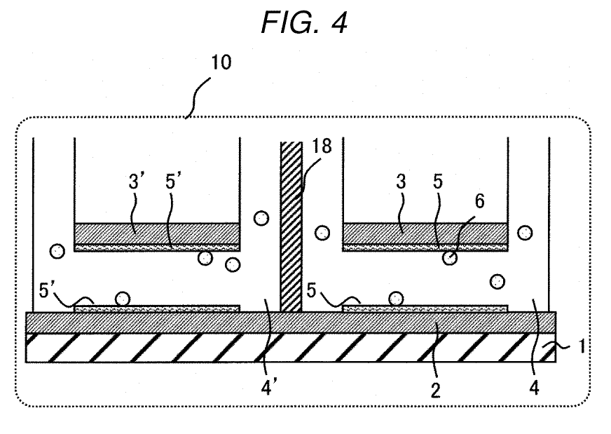

FIG. 4 is a cross-sectional view illustrating a structure of a main part of the chemical sensor 10 using the first electrode 2 as a common electrode, which is configured to include two pairs of first electrodes 2 and second electrodes 3 and 3′. A partition wall 18 partitioning the flow path 4 is configured by leaving the protective film 20 by etching. Although the mechanical strength can be improved and the flow of the sample can be adjusted by the partition wall, the partition wall can be omitted in the example illustrated in FIG. 4 and the followings. In a case where equal samples are simultaneously supplied to the plurality of chemical sensors 10, it may be preferable that there is no partition wall 18. In addition, although not illustrated in the drawings, both the left and right sides of the chemical sensor 10 can be configured similarly to the partition wall 18.

As illustrated in FIG. 4, the first electrode 2 is formed on the front surface of the semiconductor substrate 1, and serves as the common electrode. Two pairs of second electrodes 3 and 3′ are disposed to face the common first electrode 2. The two pairs of sensor elements configured to include the common first electrode 2 and the second electrodes 3 and 3′ are respectively provided with the flow paths 4 and 4′ and the chemical substance capturing portions 5 and 5′ with the same type. Since the second electrodes 3 and 3′ are connected to a common terminal via the metal wiring layer, one chemical sensor 10 is configured by parallel connection of two pairs of sensor elements. In the example of FIG. 4, the capacitances are connected in parallel, so that the capacitance to be detected can be increased.

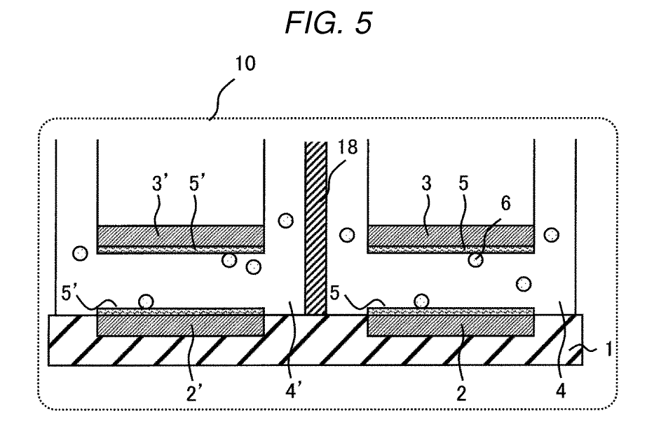

FIG. 5 is another example, and a cross-sectional view illustrating a structure of a main part of the chemical sensor 10 having two pairs of first electrodes 2 and 2′, second electrodes 3 and 3′, the flow path 4, and the chemical substance capturing portions 5 and 5′ on the common semiconductor substrate 1.

As illustrated in FIG. 5, two pairs of first electrodes 2 and 2′ are separately formed on the common semiconductor substrate 1, and second electrodes 3 and 3′ corresponding thereto, flow paths 4 and 4′, and chemical substance capturing portions 5 and 5′ with the same type may be configured to be provided. In this case as well, as in FIG. 4, since the second electrodes 3 and 3′ are connected to the common terminal via the metal wiring layer, one chemical sensor 10 is configured by parallel connection of two pairs of sensor elements. Therefore, as in the example of FIG. 4, the capacity to be detected can be increased.

In the configuration of FIG. 5, a patterning process for separately forming the first electrodes 2 and 2′ is added, but since the first electrodes 2 and 2′ are formed only in regions facing the second electrodes 3 and 3′, unnecessary parasitic capacitance can be suppressed, and a structure suitable for electrostatic capacitance design of the chemical sensor 10 is obtained.

(2) In Example 1, the chemical sensor is configured to include the first electrode and the second electrode which are disposed to face each other, but a chemical sensor with a configuration having a third electrode set to the same potential as the first electrode may be used.

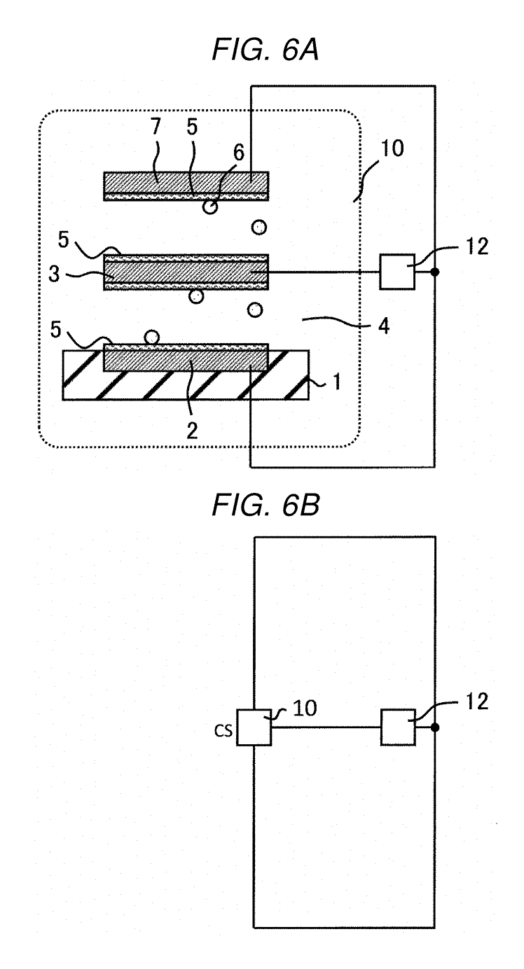

FIG. 6A illustrates an example of a chemical sensor 10 in which a first electrode 2 formed on a p-type Si semiconductor substrate 1, a second electrode 3 disposed so as to face the first electrode 2, and a third electrode 7 disposed so as to face the second electrode on the side opposite to the direction in which the first electrode is disposed with respect to the second electrode are provided. The chemical sensor 10 may have a configuration in which the third electrode 7 is formed, for example, to include n⁺-polycrystalline silicon by the CVD method, and patterned, and the flow path 4 and the chemical substance capturing portion 5 are provided.

Here, it is configured that the liquid or gas can respectively flow in between the flow path 4, the first electrode 2 and the second electrode 3, and between the second electrode 3 and the third electrode 7. In addition, the chemical substance capturing portion 5 is formed not only in the first electrode 2 and the second electrode 3 but also in the third electrode 7. Furthermore, the third electrode 7 is connected to the first electrode 2 and the common terminal via the metal wiring layer. Therefore, one chemical sensor 10 is configured by parallel connection of two pairs of sensor elements disposed one above the other, and it is configured to increase the electrostatic capacitance of the chemical sensor.

In the example of FIGS. 6A and 6B, both the distance between the first electrode 2 and the second electrode 3, and the distance between the second electrode 3 and the third electrode 7 are set to be a value smaller than approximately 100 times the size of the chemical substance 6 molecules. The chemical substance capturing portions 5 are provided on both of the facing electrodes in the above-described example, but may be provided on only one thereof. As a matter of course, the sensitivity improves when the units are provided on both.

FIG. 6B is an equivalent circuit diagram in which the chemical sensor 10 of FIG. 6A is rewritten with a simplified symbol.

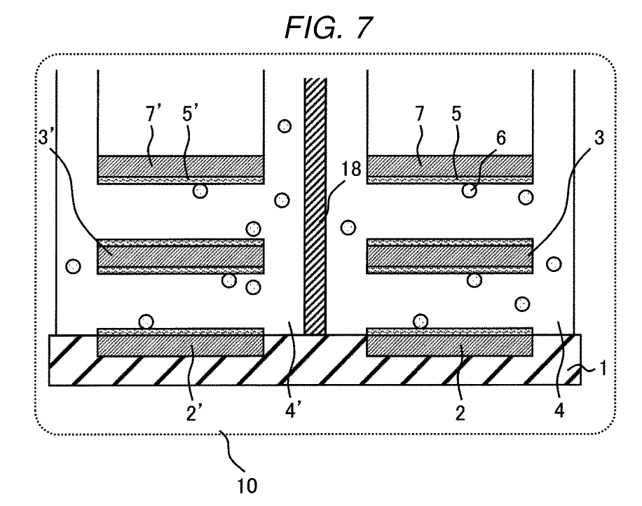

FIG. 7 illustrates an example of a chemical sensor having a combination of the above (1) and (2). In other words, the chemical sensor 10 is configured to include a plurality of pairs of the first electrode 2, the second electrode 3, and the third electrode 7. In this configuration, since the effects of increasing the individual electrostatic capacitances described above are obtained by combining, a chemical sensor having a larger electrostatic capacitance in the same area can be obtained.

(3) In addition, in Example 1, although the semiconductor substrate 1 is set to be a p-type Si, without being limited thereto, an n-type Si, an n-type silicon carbide (SiC), or a p-type SiC may be used. However, in a case where the conductivity type of the substrate is the n-type, from the viewpoint of element isolation and reduction of parasitic capacitance, it is preferable to form a semiconductor region 15 by p-type ion implantation.

(4) In addition, although the n⁺-type semiconductor region 15 formed by ion-implanting an n-type impurity into the upper surface of the p-type Si semiconductor substrate 1, and performing the activation process of the implanted impurity (annealing) is used as the first electrode 2 in Example 1, the example is not limited thereto. For example, a silicide layer formed by reacting at least a portion of the n⁺-type semiconductor region 15 with a metal may be used as the first electrode 2.

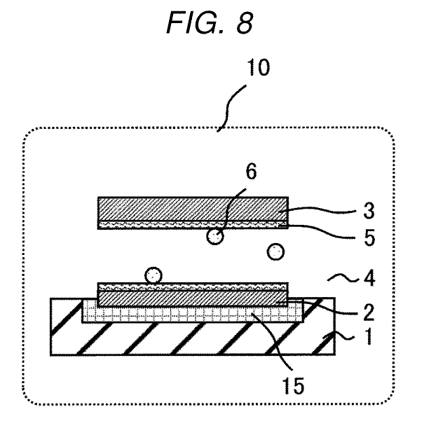

FIG. 8 is a cross-sectional view illustrating a structure of a main part of the chemical sensor in which the n⁺-type semiconductor region 15 is partially silicided to form the first electrode 2. Here, a metal for forming the silicide layer may be a metal material such as nickel, titanium, tungsten, which is generally used in the semiconductor manufacturing process.

(5) In addition, although the n⁺ polycrystalline silicon is used as the second electrode 3 in Example 1, the example is not limited thereto. For example, by using a silicon on insulator (SOI) substrate for the semiconductor substrate, monocrystallin silicon can be used as the second electrode 3. In addition, the silicide layer formed by siliciding polycrystalline silicon or monocrystallin silicon as described above may be used as the second electrode 3. Furthermore, a metal which does not disappear when the spacer is removed by etching, and which is not etched with hydrofluoric acid or has a slow etching rate, such as nickel or gold may be used as the second electrode 3.

(6) In addition, although the third electrode 7 is formed to include, for example, the n⁺ polycrystalline silicon by the CVD method in the above-described (2) of Example 1, the example is not limited thereto. For example, a silicide layer, or nickel or gold which is a metal not etched with hydrofluoric acid or having a slow etching rate may be used as the third electrode 7.

(7) In addition, although the p-type Si is used in the semiconductor substrate 1, the n⁺ semiconductor region is used in the first electrode 2, the n⁺ polycrystalline silicon is used in the second electrode 3, and the n⁺ polycrystalline silicon is used in the third electrode 7 in the above-described (2) of Example 1, the example is not limited thereto. For example, n-type Si, n-type SiC or p-type SiC may be used in the semiconductor substrate, n⁺ and p⁺ semiconductor regions or silicide formed by ion-implanting may be used in the first electrode 2, nickel or gold which is the metal not etched with hydrofluoric acid or having the slow etching rate may be used in the second electrode 3, and n⁺ and p⁺ polycrystalline silicon or silicide maybe used in the third electrode 7, respectively.

(8) In addition, as illustrated in FIGS. 4 and 5, although two pairs (plural) of sensor elements are formed on the common semiconductor substrate 1 in the above-described (1) of Example 1, and one chemical sensor 10 is configured by parallel connection of the two pairs of sensor elements, the example is not limited thereto.

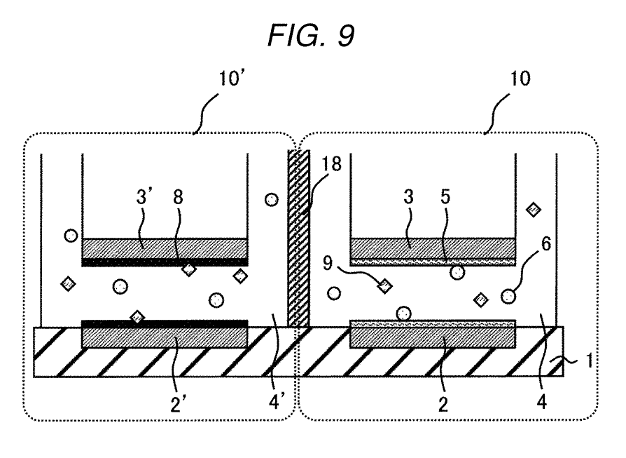

As illustrated in FIG. 9, the example may be two pairs of chemical sensors 10 and 10′ configured to include two pairs of first electrodes 2 and 2′ which are separately formed on the common semiconductor substrate 1, the second electrodes 3 and 3′ corresponding thereto, the flow paths 4 and 4′, the chemical substance capturing portions 5 and 8 with different types, and to separately measure the electrostatic capacitance. Different chemical substances 6 and 9 can be detected by mixing chemical sensors 10 and 10′ provided with different chemical substance capturing portions 5 and 8.

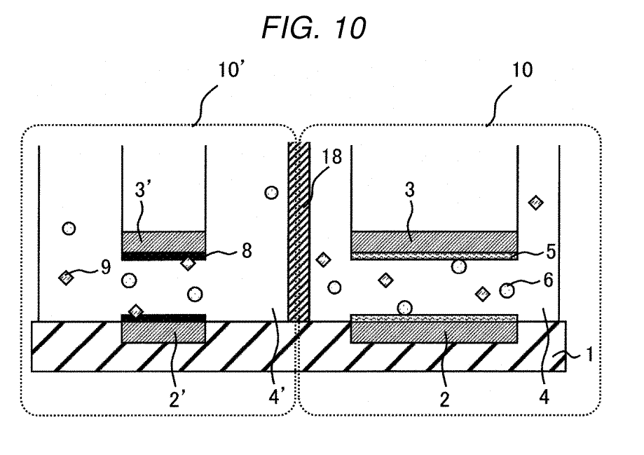

FIG. 10 illustrates another example. Although the chemical substance capturing portions 5 and 8 of the chemical sensors 10 and 10′ have substantially the same area in the example of FIG. 9, in order to adjust the signal intensity obtained from the chemical sensors 10 and 10′, and to simplify a gain adjustment in the measuring circuit, a facing area between the electrodes may be varied for each chemical sensor and 10′ provided with different chemical substance capturing portions 5 and 8.

### Operation and Effect of Example

Next, the effect according to Example 1 will be described using FIG. 1A again. As illustrated in FIG. 1A, the predetermined chemical substance 6 is captured by the chemical substance capturing portion 5 for a certain period of time and stagnates. While the prescribed chemical substance 6 is stagnant, the predetermined chemical substance 6 exists in a form in which the liquid or gas (for example, air) flowing in the flow path is partially replaced, and the dielectric constant between the first electrode 2 and the second electrode 3 which are two facing electrodes changes. As for the change in the dielectric constant, the detection signal is measured as a change in the electrostatic capacitance in the detection system 12 connected via the metal wiring.

Here, the intensity of the detection signal is determined by the size of the chemical substance 6 captured for a certain time by the chemical substance capturing portion 5, and the distance between the two facing electrodes. For example, in a case of an odorant molecule drifting in air, the molecule is a low molecular weight substance with high volatility, and the molecular weight is approximately 17 to 400 of ammonia. Therefore, in the case of the odorant molecule, the molecule is only approximately 0.1 to 2 nm in size. If the molecule is circular or elliptical, the size is a value approximated by a long diameter, and if the molecule is a string or amorphous molecule, the size is a value approximated by a length or a long side. Depending on the accuracy of the detection system, in a case of an evaluation system that can measure up to several fF as a change in minute signal intensity, and in a case where ammonia is captured on the entire surface of the chemical substance capturing portion 5 and the air in the region thereof is entirely replaced by a capacitor having the electrode size of 100 μm×100 μm and the distance between the electrodes of 10 nm, a detection signal corresponding to the replaced amount is obtained. Therefore, the odorant molecule is easily detected by setting the distance between the electrodes to approximately 100 times or less the size of the molecule to be detected. Therefore, by setting the distance D between two facing electrodes to 200 nm or less, more preferably 10 nm or less, even when the target is a substance having a small molecular weight, various types of odorant molecules can be detected, without requiring signal amplification.

In addition, it is preferable that the distance D between two facing electrodes ensures a size that functions as a flow path of the odorant molecule. When the size of the odorant molecule is approximately 0.1 to 2 nm as described above, the flow path width F is ensured approximately ten times that of the odor molecule in the direction perpendicular to the semiconductor substrate 1, and is ensured approximately 1 to 20 nm. Since the flow path width F is obtained by subtracting the thickness of the chemical substance capturing portion 5 from the distance D between the electrodes, if the thickness of the chemical substance capturing portion 5 is set to be 1 nm in a single layer, it is preferable to secure at least 2 nm as the distance D between the electrodes. It is possible to process in units of nm in the current semiconductor process, and device processing with the dimensions as described above is possible by applying a semiconductor process.

### Example 2

A structure of a chemical sensor according to Example 2 is schematically the same as that of Example 1, but the material of the chemical substance capturing portion 5 and the material of the electrode depending thereon are different therefrom.

Points different from Example 1 of the structure of the chemical sensor 10 according to Example 2 will be described with reference to FIGS. 1A and 1B. In Example 2, the semiconductor substrate 1 is set to be a general semiconductor substrate which is not etched with hydrofluoric acid or has a slow etching rate. As such a semiconductor substrate, for example, silicon (Si), silicon carbide (SiC), gallium nitride (GaN), gallium arsenide (GaAs), germanium (Ge), silicon germanium (SiGe) or the like may be used.

The first electrode 2 uses the n⁺ and p⁺ semiconductor regions formed by ion-implanting, or an alloy of a semiconductor region and a metal. The second electrode 3 uses a thiol-modifiable metal such as gold. The chemical substance capturing portion 5 uses thiol having an organic functional group on the front surface, or synthesized molecules further produced by molecular imprinting on the front surface of thiol, or molecules with modified antibody. Therefore, in a case of an electrode which cannot be modified with thiol, the chemical substance capturing portion 5 on the side of the first electrode 2 is not formed.

A modification example of Example 2 will be described with reference to FIGS. 6A and 6B. The first electrode 2, the second electrode 3, and the chemical substance capturing portion 5 are the same as in Example 1. Furthermore, the third electrode 7 uses, for example, n⁺ polycrystalline silicon by a CVD method, a silicide layer, and nickel or gold which is a metal not etched with a hydrofluoric acid or having a slow etching rate. Here, in a case of an electrode which cannot be modified with thiol as the third electrode 7, the chemical substance capturing portion 5 on the side of the third electrode 7 is not formed.

In Example 2, the material of the chemical substance capturing portion 5 and the material of the electrode depending thereon are different, but the effect thereof is obtained in the same manner as in Example 1. However, since the material of each portion is different, a more suitable method may be selected from the easiness in forming the chemical substance capturing portion 5 and easiness in handling the medicine.

### Example 3

A structure of a chemical sensor according to Example 3 is schematically the same as that of Example 1, but the material of the chemical substance capturing portion 5 and the material of the electrode depending thereon are different therefrom.

Points different from Example 1 of the structure of the chemical sensor 10 according to Example 3 will be described with reference to FIGS. 1A and 1B. In Example 3, the semiconductor substrate 1 may be a general semiconductor substrate which is not etched with hydrofluoric acid, or has a slow etching rate. The first electrode 2 uses the n⁺ and p⁺ semiconductor regions formed by ion-implanting, or an alloy of a semiconductor region and a metal. The second electrode 3 uses electrode materials generally used in semiconductor manufacturing process such as n⁺ polycrystalline silicon by a CVD method, a silicide layer, and nickel or gold which is a metal not etched with a hydrofluoric acid or having a slow etching rate. The chemical substance capturing portion 5 uses silicon nitride (Si₃N₄) or tantalum pentoxide (Ta₂O₅) which is an insulator not etched with hydrofluoric acid or having a slow etching rate.

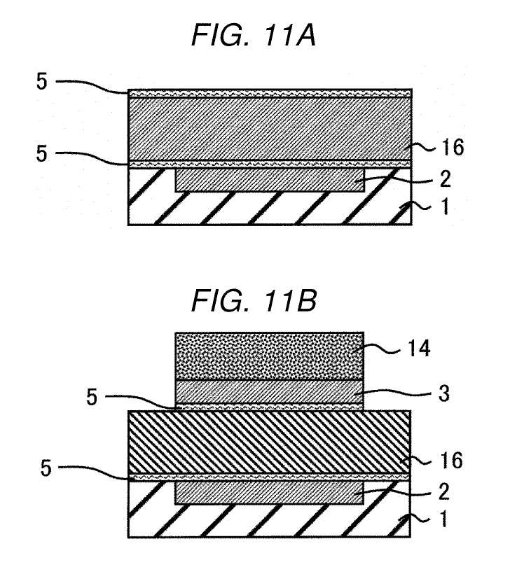

Points different from Example 1 of the manufacturing method of the chemical sensor 10 according to Example 3 will be described with reference to FIGS. 11A and 11B. The manufacturing method of the chemical sensor according to Example 3 is the same as that of Example 1 up to the intermediate process, but the manufacturing method differs from the process after forming the n⁺-type semiconductor region 15 illustrated in FIG. 2A, and after forming the first electrode 2 by removing the mask material 14.

As illustrated in FIG. 11A, after forming the first electrode 2, silicon nitride (Si₃N₄) or tantalum pentoxide, which is an insulator not etched with hydrofluoric acid or having a slow etching rate, is formed on the upper surface of the semiconductor substrate 1, as a chemical substance capturing portion 5. Next, a spacer 16 is further formed, and the chemical substance capturing portion 5 is further formed thereon.

As a subsequent process in FIG. 11B, a film to be the material of the second electrode, for example, n⁺ polycrystalline silicon by the CVD method is formed on the upper surface of the chemical substance capturing portion 5. A mask material 14 is formed on the upper surface of the n⁺ polycrystalline silicon, the mask material 14 is patterned by a photolithography technique, and thereafter the layer to be the material of the second electrode is processed to form a second electrode 3. Further, the chemical substance capturing portion 5 is similarly processed.

Next, the spacer 16 is selectively etched with hydrofluoric acid to form a flow path 4. Thereafter, if necessary, the chemical substance capturing portion 5 on the semiconductor substrate 1 is etched so that the chemical sensor having the shape illustrated in FIGS. 3A to 3C is substantially completed.

In Example 3, since the chemical substance capturing portion 5 is previously formed and there is a limit applicable to the material thereof, it is difficult to form a chemical substance capturing portion for a certain chemical substance. On the other hand, since the unit is formed by film formation prior to flow path formation, a uniform chemical substance capturing portion 5 can be obtained.

### Example 4

It is possible to configure a chemical substance detection device by using a plurality of pieces of at least one of the chemical sensors 10 described in Examples 1 to 3 described above.

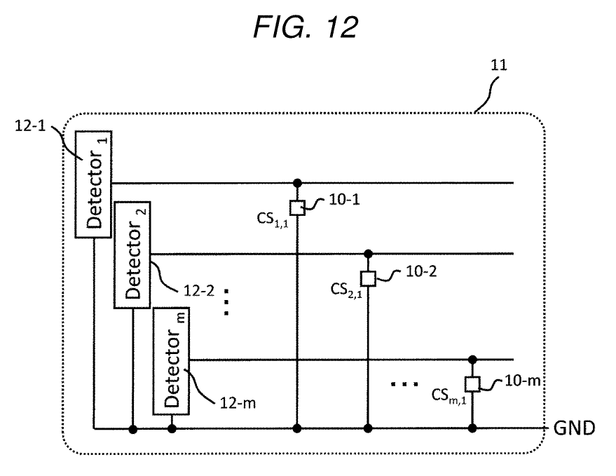

FIG. 12 is a schematic diagram illustrating a configuration of a chemical substance detection device 11 according to Example 4. As illustrated in FIG. 12, a plurality of sets are built as a set of chemical sensors 10-1 to 10-m and detection systems 12-1 to 12-m for detecting the signals thereof in the chemical substance detection device 11. These plural sets are connected to the same ground line (GND) so as to have a common potential.

One electrode of the chemical sensor 10 configuring each set is commonly connected to the ground line, and the other electrode of the chemical sensor is connected to each detection system 12. Each detection system 12 is provided with a power supply for applying a voltage and a current meter, and can detect a chemical substance by detecting an electrostatic capacitance according to a dielectric constant between the first electrode and the second electrode.

In the chemical substance detection device 11 according to Example 4, for example, each chemical sensor 10 is spatially widely disposed, and thus the spatial distribution of a predetermined chemical substance can be measured.

In addition, the plurality of chemical sensors 10-1 to 10-m use the same types of sensors in the example of FIG. 12. As a modification example of Example 4, it is possible to use different types of sensors in the plurality of chemical sensors 10-1 to 10-m. That is, the type of the predetermined chemical substance 6 captured by the chemical substance capturing portion 5 is different. Therefore, it is possible to simultaneously measure a plurality of types of chemical substances 6. This principle is the same as previously described in FIG. 9.

In addition, in order to adjust signal intensities obtained from the chemical sensors 10-1 to 10-m, the facing area between the electrodes may be changed for each chemical sensor 10 provided with different chemical substance capturing portions 5 as another modification example of Example 4. This principle is the same as previously described in FIG. 10.

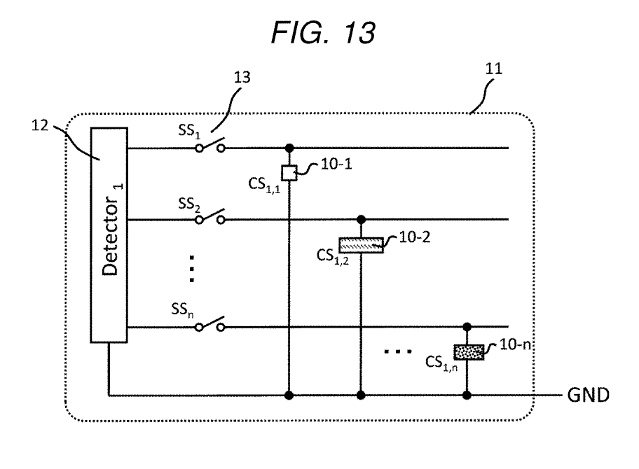

A further modification example of Example 4 will be described with reference to FIG. 13. In FIG. 13, a plurality of chemical sensors 10-1 to 10-n in which the types of predetermined chemical substances 6 captured by the chemical substance capturing portion 5 are different, and the facing areas of the electrodes are different are connected to the detection system 12 via a selection switch 13. The selection switch 13 may switch on/off the electrical connection, and uses, for example, a transistor, micro electro mechanical systems (MEMS) switch, or the like. In the modification example of Example 4, since the plurality of chemical sensors 10-1 to 10-n are connected to one detection system 12 via the selection switch 13, the chemical substance detection device 11 can be downsized. In addition, the facing areas between the electrodes of the chemical sensors 10-1 to 10-n are changed, and thus it is easy to adjust the output level and process in one detection system 12.

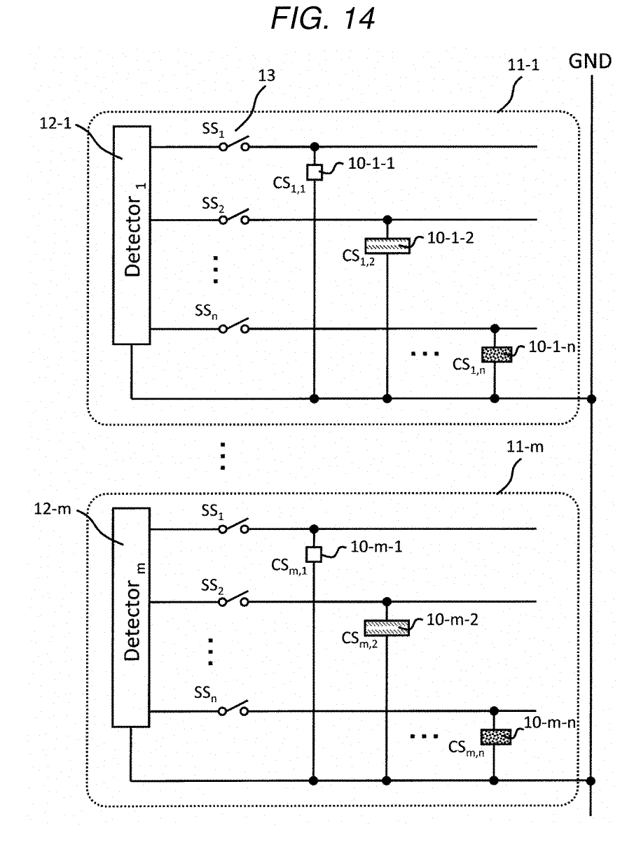

A further modification example of Example 4 will be described with reference to FIG. 14. In FIG. 14, a plurality of chemical substance detection devices 11-1 to 11-m in which a plurality of chemical sensors 10 having different chemical substance capturing portions 5 described above are connected to a detection system 12 via a selection switch 13 are included. In the modified example of Example 4, a plurality of types of predetermined chemical substances 6 can be simultaneously measured including the spatial distribution by the m number of detection systems 12-1 to 12-m.

In the chemical sensor of the invention, since the distance between the first electrode and the second electrode for detecting the change in the dielectric constant is configured to have a value smaller than the predetermined ratio (approximately 100 times) with respect to the size of the molecule of the predetermined chemical substance to be detected, it is possible to detect a change in the dielectric constant between the facing electrodes without requiring signal amplification.

Hereinbefore, although the invention made by the inventor is specifically described based on the embodiments, it is needless to say that the invention is not limited to the embodiments described above, and various modifications can be made without departing from the gist thereof.

What is claimed is:

1. A chemical sensor comprising:
a semiconductor substrate of a first conductivity type;
a first electrode that is formed on a front surface of the semiconductor substrate;
a second electrode that is disposed to face the first electrode in a vertical direction;
a flow path in which a liquid or a gas can flow between the first electrode and the second electrode; and
a chemical substance capturing portion that is disposed in at least a partial region between the first electrode and the second electrode in the flow path, and bonded with a predetermined chemical substance,
wherein a distance between the first electrode and the second electrode is set to be 2 nm or more and 200 nm or less, and
a change in dielectric constant between the first electrode and the second electrode is detected.

2. The chemical sensor according to claim 1,
wherein the first electrode is configured as a common electrode,
the second electrode is configured as a plurality of second electrodes that are formed apart from each other,
the first electrode is set to be a first potential, and the plurality of second electrodes are set to be second potentials, and
an electrostatic capacitance between the first electrode and the plurality of second electrodes is detected.

3. The chemical sensor according to claim 1,
wherein the first electrode is configured as a plurality of first electrodes on the front surface of the semiconductor substrate,
the second electrode is configured as a plurality of the second electrodes that are disposed to face the plurality of the first electrode in the vertical direction,
the plurality of the first electrodes are set to be the first potentials,
the plurality of the second electrodes are set to be the second potentials, and
the electrostatic capacitances between the plurality of first electrodes and the plurality of second electrodes are detected.

4. The chemical sensor according to claim 1, further comprising:
a third electrode that is disposed to face the second electrode in a vertical direction in a direction opposite to a direction in which the first electrode is disposed with respect to the second electrode,
wherein a distance between the second electrode and the third electrode is set to be 2 nm or more and 200 nm or less, and
the third electrode is wired so as to be a voltage equal to that of the first electrode.

5. The chemical sensor according to claim 1,
wherein the semiconductor substrate is formed of a material selected from silicon, silicon carbide, gallium nitride, gallium arsenide, germanium, and silicon germanium, and
the first electrode is formed of a semiconductor layer of a second conductivity type that is formed on the front surface of the semiconductor substrate or an alloy of at least a portion of the semiconductor substrate and a metal.

6. The chemical sensor according to claim 5,
wherein the second electrode is formed of a material selected from monocrystalline silicon, polycrystalline silicon, silicide, nickel, and gold, and
the chemical substance capturing portion is at least one selected from silicon nitride, tantalum pentoxide, a silane coupling agent, thiol, and a synthetic molecule prepared by molecular imprinting.

7. The chemical sensor according to claim 1,
wherein the flow path
introduces the chemical substance between the first electrode and the second electrode in a direction perpendicular to the semiconductor substrate,
passes the chemical substance through between the first electrode and the second electrode, and
discharges the chemical substance from between the first electrode and the second electrode in the direction perpendicular to the semiconductor substrate.

8. The chemical sensor according to claim 1,
wherein the first electrode is configured as a plurality of first electrodes that includes a first electrode A and a first electrode B on the front surface of the semiconductor substrate,
the second electrode is configured as a plurality of second electrodes that includes a second electrode A and a second electrode B which are disposed to face the plurality of first electrodes in the vertical direction,
a first chemical substance capturing portion is formed on at least a portion of the first electrode,
a second chemical substance capturing portion is formed on at least a portion of the second electrode,
the first electrode A and the second electrode A face each other, and a facing area between the electrodes thereof is an area A,
the first electrode B and the second electrode B face each other, and a facing area between the electrodes thereof is an area B,
the first chemical substance capturing portion and the second chemical substance capturing portion capture different substances, and
the area A and area B are different from each other.

9. The chemical sensor according to claim 1,
wherein the distance between the first electrode and the second electrode is 10 nm or less.

10. A chemical substance detection method comprising:
using a semiconductor substrate;
using a first electrode that is formed on a front surface of the semiconductor substrate;
using a second electrode that is disposed to face the first electrode in a vertical direction;
using a chemical substance capturing portion that is disposed in at least a partial region between the first electrode and the second electrode, and bonded with a predetermined chemical substance;
setting a distance between the first electrode and the second electrode to be 100 times or less the size of the chemical substance;
supplying a gas or a liquid containing the chemical substance, between the first electrode and the second electrode;
detecting a change in dielectric constant between the first electrode and the second electrode by capturing the chemical substance in the chemical substance capturing portion; and
detecting the chemical substance based on the change in the dielectric constant.

11. The chemical substance detection method according to claim 10,
wherein the first electrode, the second electrode, and a flow path for supplying a gas or a liquid containing the chemical substance are prepared by a semiconductor manufacturing process.

12. The chemical substance detection method according to claim 11,
wherein a distance between the first electrode and the second electrode is set to be 2 nm or more and 200 nm or less.

13. A chemical substance detection device comprising:
a plurality of chemical sensors, each of which includes
a semiconductor substrate;
a first electrode that is formed on a front surface of the semiconductor substrate;
a second electrode that is disposed to face the first electrode in a vertical direction;
a flow path in which a liquid or a gas can flow between the first electrode and the second electrode;
a chemical substance capturing portion that is disposed in at least a partial region between the first electrode and the second electrode in the flow path, and bonded with a predetermined chemical substance; and
in which a distance between the first electrode and the second electrode is set to be 2 nm or more and 200 nm or less,
wherein one electrode of the first or second electrode of the chemical sensor is connected to ground,
the other electrode of the chemical sensor is connected to a detection system, and
the detection system includes a power supply for applying a voltage and a current meter, and detects an electrostatic capacitance between the first electrode and the second electrode.

14. The chemical substance detection device according to claim 13,
wherein at least two types or more of different chemical sensors of the chemical substance capturing portion are included, and
each chemical sensor having the different chemical substance capturing portion has a different electrode size.

15. The chemical substance detection device according to claim 14,
wherein a plurality of the other electrodes are connected to the same detection system via selection switches, and
only the information on the chemical sensor that is selected by the selection switch is selectively output.
